(19) United States
(12) Patent Application Publication
(10) Pub. No.: US 2026/0257002 A1
(43) Pub. Date: Sep. 3, 2026

(54) SYSTEM FOR NEGATIVE PRESSURE WOUND THERAPY

(71) Applicant: Solventum Intellectual Properties Company, Maplewood, MN (US)

(72) Inventors: Andrew J. MCGREGOR, San Antonio, TX (US); Tyler H. SIMMONS, San Antonio, TX (US); Larry T. RANDOLPH, San Antonio, TX (US); Michael R. OLIVA, San Antonio, TX (US); Richard M. KAZALA, San Antonio, TX (US); Jonathan G. REHBEIN, San Antonio, TX (US); Michael POLLOCK, San Antonio, TX (US)

(21) Appl. No.: 19/154,671

(22) PCT Filed: Feb. 13, 2024
(86) PCT No.: PCT/IB2024/051317

§ 371 Date: Aug. 7, 2025

Related U.S. Application Data
(60) Provisional application No. 63/445,011, filed on Feb. 13, 2023.

(51) Int. Cl. A61M 1/00; A61F 13/05

(52) U.S. Cl. CPC A61M 1/913; A61F 13/05; A61M 1/962; A61M 1/984

(57) ABSTRACT

A system for negative pressure wound therapy (NPWT) includes a bridge dressing including a first bridge end portion proximal to the wound and a second bridge end portion opposite to the first bridge end portion. The system further includes a pump and a casing spaced apart from the wound. The casing includes a first portion including a first inner cavity configured to at least partially receive the pump therein, a second inner cavity spaced apart from the first inner cavity, and a channel fluidly communicating the first inner cavity with the second inner cavity. The casing further includes a second portion pivotally connected to the first portion and a third portion pivotally connected to the first portion and pivotable relative to the first portion independent of the second portion.

### CROSS-REFERENCE TO RELATED APPLICATIONS

This application is a U.S. National Stage Entry of PCT International Application No. PCT/IB2024/051317, filed Feb. 13, 2024, which claims the benefit of priority to U.S. Provisional Application No. 63/445,011, filed on Feb. 13, 2023, which is incorporated herein by reference in its entirety.

### TECHNICAL FIELD

The present disclosure relates generally to negative pressure wound therapy. More particularly, the present disclosure relates to a system for negative pressure wound therapy of a wound.

### BACKGROUND

Negative pressure wound therapy (NPWT) systems are embodied as sealed wound-care systems particularly indicated for chronic persistent wounds and/or complicated wounds. Specifically, for promoting wound healing, a pressure that is reduced relative to the surroundings (commonly referred to as “negative pressure”) is applied to the wound. The negative pressure causes mechanical contraction of the wound and removal of exudates, such as, slough, necrotic tissue, microbial load (e.g., bacteria and biofilms) from the wound, thus promoting formation of granulation tissues and accelerating wound healing. In NPWT systems, the negative pressure is usually generated by an electromechanical pump which displaces the air within a sealed volume around the wound. However, conventional NPWT systems may be onerous to wear for ambulatory patients due to large rigid therapy units and stiff tubing connecting a dressing and the electromechanical pump. This may lead to a negative experience for a patient. Therefore, there is a need for a system to provide NPWT to a wound by overcoming the above-mentioned limitations.

### SUMMARY

In a first aspect, the present disclosure provides a system for negative pressure wound therapy (NPWT) of a wound. The system includes a bridge dressing including a first bridge end portion proximal to the wound and a second bridge end portion opposite to the first bridge end portion. The system further includes a pump and a casing spaced apart from the wound. The casing includes a first portion including a first inner cavity configured to at least partially receive the pump therein, a second inner cavity spaced apart from the first inner cavity and configured to receive the second bridge end portion of the bridge dressing therein, and a channel fluidly communicating the first inner cavity with the second inner cavity. The casing further includes a second portion pivotally connected to the first portion and configured to at least partially engage the first portion in a closed position of the second portion. The second portion includes a third inner cavity that is aligned with the first inner cavity in the closed position of the second portion, such that the pump is received within the first inner cavity and the third inner cavity. The casing further includes a third portion pivotally connected to the first portion and pivotable relative to the first portion independent of the second portion. The third portion is configured to at least partially engage the first portion in a closed position of the third portion. The third portion covers the second inner cavity in the closed position of the third portion, such that the second bridge end portion of the bridge dressing is received between the second inner cavity and the third portion.

In a second aspect, the present disclosure provides a system for negative pressure wound therapy (NPWT) of a wound. The system includes a bridge dressing including a first bridge end portion proximal to the wound and a second bridge end portion opposite to the first bridge end portion. The system further includes a pump and a casing spaced apart from the wound. The casing includes an inner cavity receiving the pump therein, an outer surface, and a channel at least partially disposed on the outer surface and fluidly communicating with the inner cavity. The second bridge end portion of the bridge dressing is connected to and disposed on the outer surface of the casing. The second bridge end portion fluidly communicates with and covers the channel.

In a third aspect, the present disclosure provides a system for negative pressure wound therapy (NPWT) of a wound. The system includes a bridge dressing including a first bridge end portion proximal to the wound and a second bridge end portion opposite to the first bridge end portion. The system further includes a tube including a first tube end portion and a second tube end portion opposite to the first tube end portion. The system further includes a first manifold disposed around the first tube end portion and fluidly coupled to the tube. The first manifold is connected to the second bridge end portion of the bridge dressing. The system further includes a second manifold disposed around the second tube end portion and fluidly coupled to the tube. The system further includes a pump and a casing spaced apart from the wound. The casing includes an inner cavity receiving the pump therein, an outer surface, and a channel at least partially disposed on the outer surface and fluidly communicating with the inner cavity. The second manifold is connected to and disposed on the outer surface of the casing. The second manifold fluidly communicates with and covers the channel.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic top view of a system for negative pressure wound therapy (NPWT) of a wound, according to an embodiment of the present disclosure;

FIG. 2A is a top perspective view of a casing of the system of FIG. 1, according to an embodiment of the present disclosure;

FIG. 2B is a rear perspective view of the casing of FIG. 2A, according to an embodiment of the present disclosure;

FIG. 2C is a side perspective view of the casing of FIG. 2A, according to an embodiment of the present disclosure;

FIG. 2D is a front perspective view of the casing of FIG. 2A, according to an embodiment of the present disclosure;

FIG. 2E is an enlarged view of a portion of the casing shown in FIG. 2A, according to an embodiment of the present disclosure;

FIG. 2F is a side sectional view of the portion of FIG. 2E along a line A-A', according to an embodiment of the present disclosure;

FIG. 2G is an enlarged view of a portion of the casing shown in FIG. 2C, according to an embodiment of the present disclosure;

FIG. 3 is a schematic top view of a system for NPWT of a wound, according to another embodiment of the present disclosure;

FIG. 4 is a schematic view of a system for NPWT of a wound, according to another embodiment of the present disclosure;

FIG. 5A is a top perspective view of a casing of the system of FIG. 1, according to another embodiment of the present disclosure;

FIG. 5B is a rear perspective view of the casing of FIG. 5A, according to an embodiment of the present disclosure;

FIG. 5C is a side perspective view of the casing of FIG. 5A, according to an embodiment of the present disclosure;

FIG. 5D is a front perspective view of the casing of FIG. 5A, according to an embodiment of the present disclosure;

FIG. 5E is an enlarged view of a portion of the casing shown in FIG. 5A, according to an embodiment of the present disclosure;

FIG. 5F is a side sectional view of the portion of FIG. 5E along a line B-B', according to an embodiment of the present disclosure;

FIG. 5G is an enlarged view of a portion of the casing shown in FIG. 5C, according to an embodiment of the present disclosure;

FIG. 6A is a top perspective view of a casing of the system of FIG. 1, according to another embodiment of the present disclosure;

FIG. 6B is a rear perspective view of the casing of FIG. 6A, according to an embodiment of the present disclosure;

FIG. 6C is a side perspective view of the casing of FIG. 6A, according to an embodiment of the present disclosure; and

FIG. 6D is a front perspective view of the casing of FIG. 6A, according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “same” or “similar” as a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” “coupled”, and variations thereof, are used broadly and encompass both direct physical connections, or indirect physical connections between two or more components that are connected together by one or more additional components. For example, a first component may be coupled to a second component by being directly connected together or by being connected by a third component. In some examples, coupling, connection, and attachment may also include mechanical, thermal, electrical, or chemical coupling (such as a chemical bond) in some contexts.

As used herein, the terms “layer,” “sheet,” and “dressing,” or variations thereof, are used to describe an article having a thickness that is small relative to its length and width.

As used herein, the term “negative pressure” broadly refers to a pressure lower than a local ambient pressure, such as an ambient pressure, in a local environment outside the sealed treatment environment provided by a dressing. In many cases, the local ambient pressure can also be the atmospheric pressure at which a wound site is located. Alternatively, the pressure can be less than the hydrostatic pressure associated with the tissue at the wound site. Unless otherwise specified, the pressure values described herein are gauge pressures. Similarly, a reference to an increase in negative pressure typically refers to a decrease in absolute pressure, while a decrease in negative pressure typically refers to an increase in absolute pressure.

As used herein, the term “wounds” may include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

Conventionally, a negative pressure wound therapy system (NPWT) system is used to promote wound healing. The NPWT system includes a therapy device configured to provide negative pressure wound therapy by reducing a pressure at a wound. The therapy device can draw a vacuum (relative to atmospheric pressure) at the wound by removing wound exudate, air, and other fluids from the wound. The fluids removed from the wound may be collected within an exudate canister. The therapy device may also extract fluids (e.g., an instillation fluid) from the wound that may be previously delivered to the wound. In some cases, in order to heal a wound, a preset volume of the instillation fluid is delivered at a wound site along with an application of the negative pressure. The instillation fluid can include, for example, a cleansing fluid, a prescribed fluid, a medicated fluid, an antibiotic fluid, or any other type of fluid which can be delivered to the wound during wound treatment. The instillation fluid may be held in an instillation canister and controllably dispensed to the wound via a tubing. Further, the NPWT system includes a wound dressing that is placed at the wound and connected in fluid communication with the instillation canister and the exudate canister.

Conventional NPWT systems may be onerous to wear for ambulatory patients due to large rigid therapy units and stiff tubing connecting a dressing and an electromechanical pump. This may lead to a negative experience for a patient. Further, in the conventional NPWT systems, attachment of the tubing with smaller sized dressings may be challenging in some applications. Moreover, a particular NPWT system may be comfortable to and preferred by some patients, and the same NPWT system may be uncomfortable to some other patients depending on the location of the wound and a patient's lifestyle and preferences.

The present disclosure provides a system for negative pressure wound therapy (NPWT) of a wound. The system includes a bridge dressing including a first bridge end portion proximal to the wound and a second bridge end portion opposite to the first bridge end portion. The system further includes a pump and a casing spaced apart from the wound. The casing includes a first portion including a first inner cavity configured to at least partially receive the pump therein, a second inner cavity spaced apart from the first inner cavity and configured to receive the second bridge end portion of the bridge dressing therein, and a channel fluidly communicating the first inner cavity with the second inner cavity. The casing further includes a second portion pivotally connected to the first portion and configured to at least partially engage the first portion in a closed position of the second portion. The second portion includes a third inner cavity that is aligned with the first inner cavity in the closed position of the second portion, such that the pump is received within the first inner cavity and the third inner cavity. The casing further includes a third portion pivotally connected to the first portion and pivotable relative to the first portion independent of the second portion. The third portion is configured to at least partially engage the first portion in a closed position of the third portion. The third portion covers the second inner cavity in the closed position of the third portion, such that the second bridge end portion of the bridge dressing is received between the second inner cavity and the third portion.

As the pump is received in the first inner cavity and the second bridge end portion of the bridge dressing is received between the second inner cavity and the third portion, the system of the present disclosure is a compact therapy unit for reduced pressure therapy of wounds of various sizes. The system of the present disclosure may be beneficial and comfortable to wear for ambulatory patients.

As the third portion is pivotally connected to the first portion and pivotable relative to the first portion independent of the second portion, the third portion may be moved pivotally relative to the first portion between an open position and the closed position independent of the second portion. Due to this, a clinician may easily replace a used bridge dressing with a new bridge dressing without requiring any access to the pump in the casing.

In some embodiments, the casing is a one-piece component made from a closed-cell foam. In an application, the casing may be web processed from a soft closed cell foam material to reduce cost, thereby providing a soft housing for the pump. The soft material of the casing may allow the casing to conform around the second bridge end portion of the bridge dressing.

In some embodiments, the first portion is adhesively bonded to the second portion in the closed position of the second portion. In some embodiments, the first portion is adhesively bonded to the third portion in the closed position of the third portion. As the second bridge end portion of the bridge dressing is received between the second inner cavity and the third portion, the bridge dressing may be cut to a desired length based on different application attributes and preferences of a patient. Moreover, the system of the present disclosure may be wearable with placement of the casing near the wound.

In some embodiments, the system further includes a wound dressing configured to be disposed at a wound site of the wound. The bridge dressing extends from the wound dressing. The first bridge end portion of the bridge dressing is connected to the wound dressing. In some embodiments, the wound dressing is integral with the bridge dressing. Therefore, there is no need for a coupler to connect the bridge dressing to the wound dressing, especially in case of smaller wounds. In some embodiments, the system of the present disclosure may further include an adhesive patch to secure the casing to body of a patient.

Therefore, the system of the present disclosure may be comfortable for different patients as the casing including the pump could be adhered to the body of the patient or could be disposed remote from the body of the patient.

The replacement of conventional tubing with the bridge dressing may also improve an overall flexibility of the system of the present disclosure and this may lead to a positive experience for a patient.

The present disclosure further provides a system for negative pressure wound therapy (NPWT) of a wound. The system includes a bridge dressing including a first bridge end portion proximal to the wound and a second bridge end portion opposite to the first bridge end portion. The system further includes a tube including a first tube end portion and a second tube end portion opposite to the first tube end portion. The system further includes a first manifold disposed around the first tube end portion and fluidly coupled to the tube. The first manifold is connected to the second bridge end portion of the bridge dressing. The system further includes a second manifold disposed around the second tube end portion and fluidly coupled to the tube. The system further includes a pump and a casing spaced apart from the wound. The casing includes an inner cavity receiving the pump therein, an outer surface, and a channel at least partially disposed on the outer surface and fluidly communicating with the inner cavity. The second manifold is connected to and disposed on the outer surface of the casing. The second manifold fluidly communicates with and covers the channel.

Such a system may be beneficial for patients who desire remote placement of the casing from their body, or for applications which require the remote placement of the casing from the body of the patients. In some embodiments, the first manifold and the second manifold may be made from a soft injection molded material or web-processed soft closed cell foam. This may provide a positive patient experience.

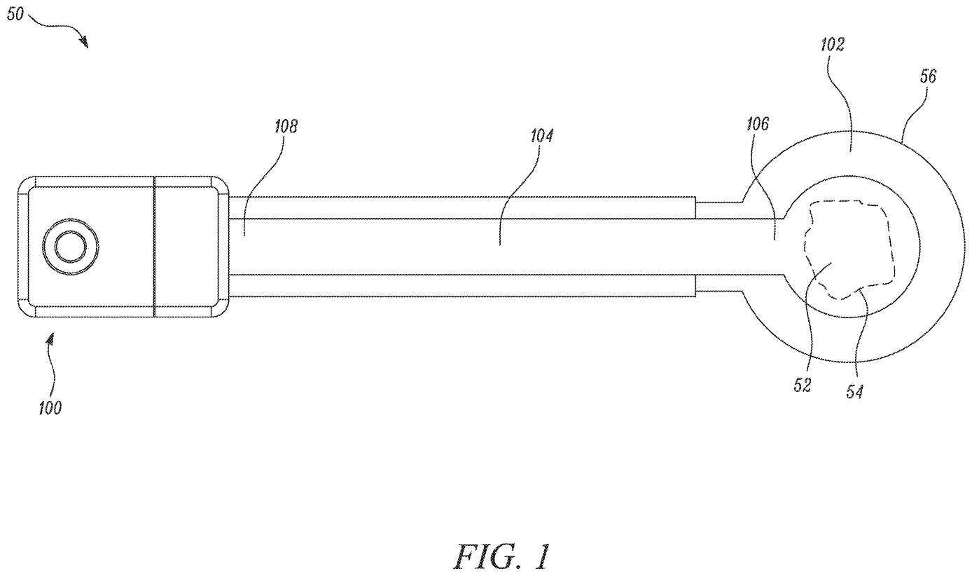

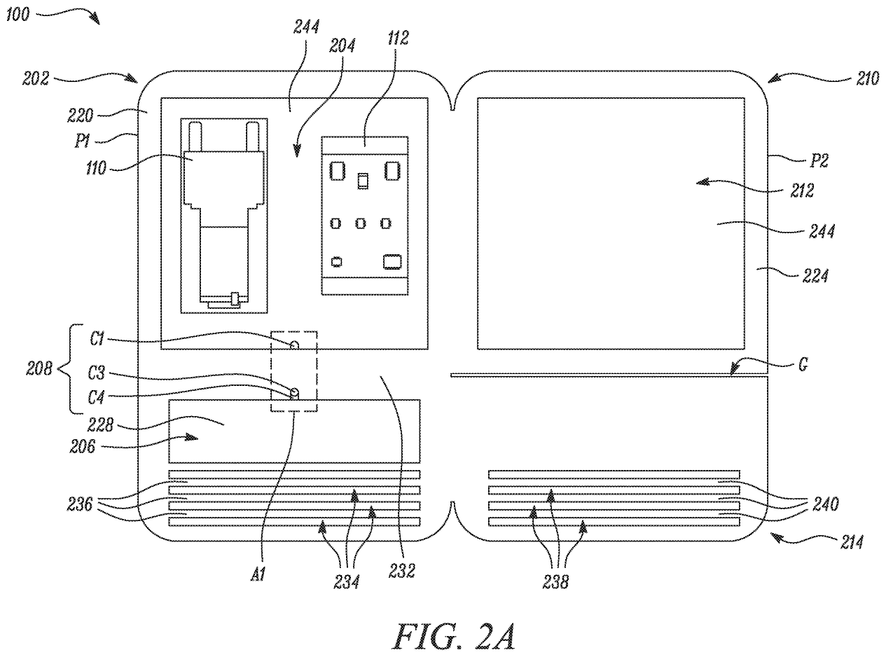

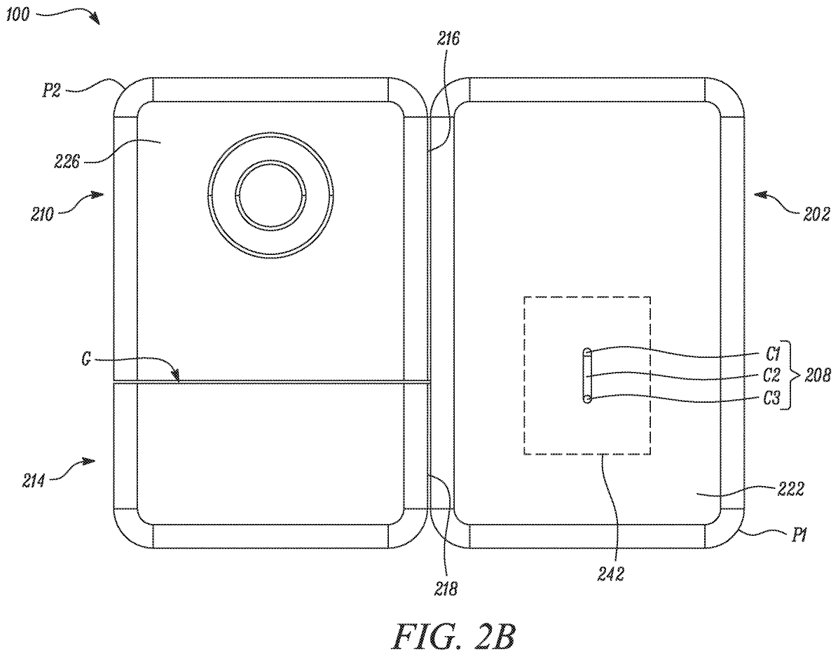

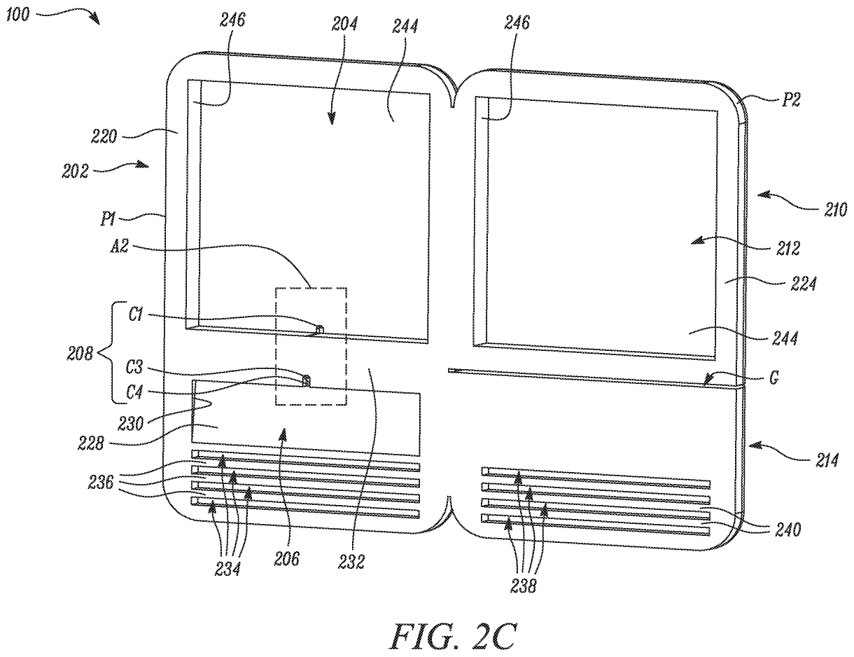

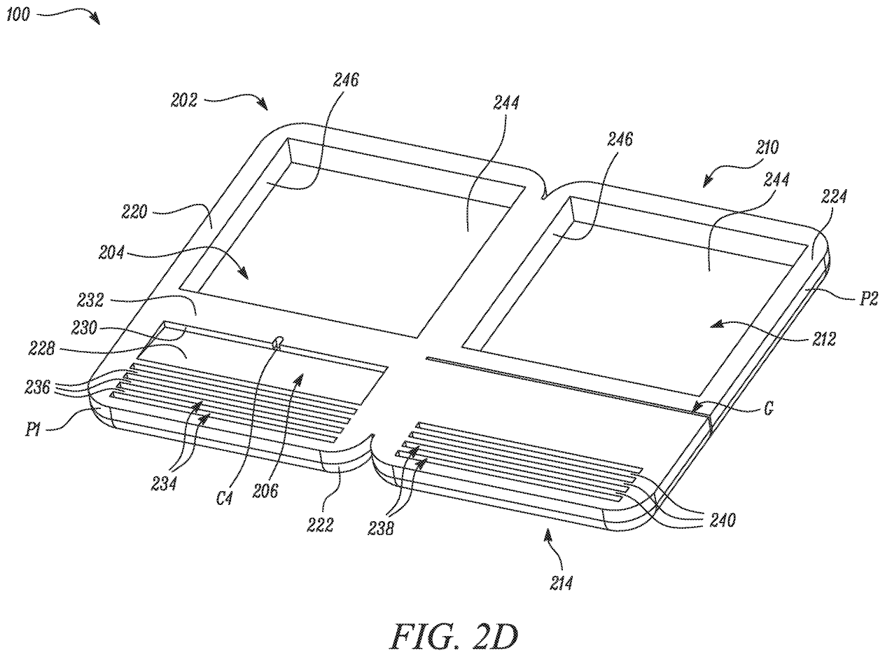

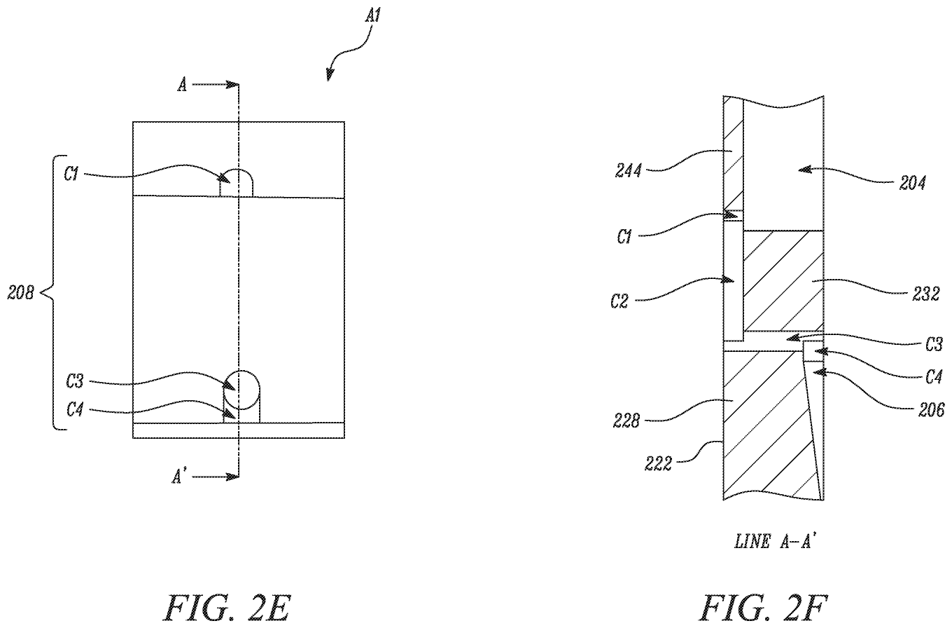

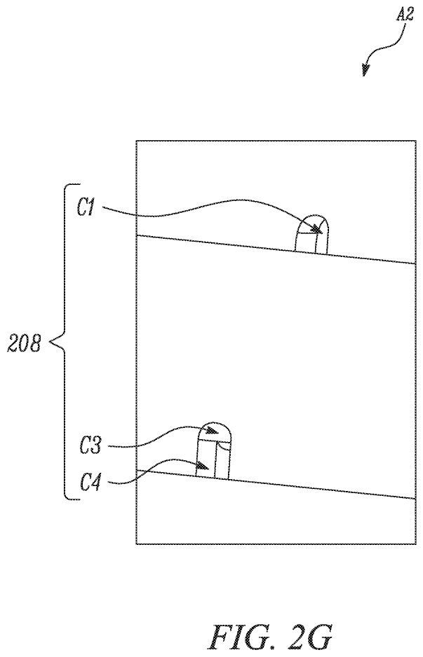

Referring now to Figures, FIG. 1 is a schematic perspective view of a system 50 for negative pressure wound therapy (NPWT) of a wound 52 of a user (not shown), according to an embodiment of the present disclosure. The system 50 is used to heal the wound 52 which is through skin (or generally epidermis) of the user. The system 50 includes a pump 110 (shown in FIG. 2A) configured to generate a negative pressure for NPWT of the wound 52.

The system 50 further includes a wound dressing 102 configured to be disposed at a wound site 54 of the wound 52. In some embodiments, the wound dressing 102 may be an advanced wound care dressing which can be suitable for healing larger wounds. The wound dressing 102 defines a sealed volume (not shown) around the wound 52. The wound dressing 102 may include a foam pad (not shown) placed at the wound 52. The foam pad may be cut such that a shape of the wound 52 is similar to a shape of the foam pad and the foam pad may be contained at the wound site 54. It should be noted that the foam pad may be replaced by any other manifolding material, such as, a micro replicated polyurethane, a gauze, a nonwoven material, etc.

The wound dressing 102 may include a sealing member 56 placed on the foam pad to cover the foam pad and the wound site 54. The sealing member 56 may include an adhesive layer (not shown) and a release liner (not shown) disposed on the adhesive layer. The release liner may protect the adhesive layer from contaminants, such as dust, debris, and the like, prior to use of the sealing member 56. The release liner may be peeled off from the adhesive layer prior to removable securement of the sealing member 56 at the wound 52. Further, the adhesive layer may include an acrylic adhesive, a silicone-based adhesive, a hydrocolloid adhesive, and a synthetic rubber adhesive. The foam pad and the sealing member 56 may include any shape and size so as to cover and enclose the wound 52 in an appropriate manner. In some embodiments, the wound dressing 102 may further include a contact layer (not shown) which may be placed at the wound 52 prior to placing the foam pad at the wound 52 to avoid a direct contact between the wound 52 and the foam pad. It should be noted that details pertaining to the wound dressing 102 as provided herein are exemplary in nature and the wound dressing 102 may include any other design/combination of materials.

The sealing member 56 is generally formed from a flexible sheet. The sealing member 56 may be formed from any material that provides a fluid seal. The sealing member 56 may, for example, be an impermeable or semi-permeable, elastomeric material. “Elastomeric” means having the properties of an elastomer. Elastomeric generally refers to a polymeric material that has rubber-like properties. More specifically, most elastomers have ultimate elongations greater than 100% and a significant amount of resilience. The resilience of a material refers to the material's ability to recover from an elastic deformation. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, EVA film, co-polyester, and silicones. Specific examples of sealing member materials include a silicone drape, 3M Tegaderm® drape, acrylic drape such as one available from Avery Dennison, or an incise drape.

The system 50 further includes a bridge dressing 104 extending from the wound dressing 102. The bridge dressing 104 includes a first bridge end portion 106 connected to the wound dressing 102 and a second bridge end portion 108 opposite to the first bridge end portion 106. The first bridge end portion 106 is proximal to the wound 52. In some embodiments, the wound dressing 102 is integral with the bridge dressing 104. In other words, the bridge dressing 104 and the wound dressing 102 together form a single component. Therefore, there is no need for a coupler to connect the bridge dressing 104 to the wound dressing 102. This may be useful in some applications, especially in case of smaller wounds. In some embodiments, the bridge dressing 104 has a flat tubular shape. In some other embodiments, the bridge dressing 104 and the wound dressing 102 are separate components connected to each other by a coupler. In some embodiments, the bridge dressing 104 is made of a material that prevents collapse upon application of the negative pressure for NPWT of the wound 52. In some embodiments, the material of the bridge dressing 104 may be non-woven, foam, micro-replicated polyurethane, and the like.

The system 50 further includes a casing 100 spaced apart from the wound 52 and the wound dressing 102. FIGS. 2A to 2D are different views of the casing 100, according to an embodiment of the present disclosure. FIG. 2E is an enlarged view of a portion A1 (shown in FIG. 2A) of the casing 100, according to an embodiment of the present disclosure. FIG. 2F is a side sectional view of the portion A1 along a line A-A′ shown in FIG. 2E, according to an embodiment of the present disclosure. FIG. 2G is an enlarged view of a portion A2 (shown in FIG. 2C) of the casing 100, according to an embodiment of the present disclosure.

Referring to FIGS. 1 to 2G, the casing 100 includes a first portion 202 including a first inner cavity 204 configured to at least partially receive the pump 110 therein. For illustration purposes, the pump 110 is shown only in FIG. 2A. The first portion 202 further includes a second inner cavity 206 spaced apart from the first inner cavity 204 and configured to receive the second bridge end portion 108 of the bridge dressing 104 therein. The first portion 202 further includes a channel 208 fluidly communicating the first inner cavity 204 with the second inner cavity 206. The channel 208 will be discussed in detail later in the description. In some embodiments, the first inner cavity 204 can be interchangeably referred to herein as “an inner cavity 204”. Therefore, the casing 100 includes the inner cavity 204 receiving the pump 110 therein.

The casing 100 further includes a second portion 210 pivotally connected to the first portion 202 and configured to at least partially engage the first portion 202 in a closed position of the second portion 210. The closed position of the second portion 210 is shown in FIG. 1. In FIGS. 2A to 2D, the closed position of the second portion 210 is not shown for illustrative purposes. The second portion 210 includes a third inner cavity 212 that is aligned with the first inner cavity 204 in the closed position of the second portion 210, such that the pump 110 is received within the first inner cavity 204 and the third inner cavity 212.

In the closed position of the second portion 2120, the pump 110 is received and sealed within the first inner cavity 204 and the third inner cavity 212, such that an outlet port (not shown) of the pump 110 is in fluid communication with ambient and an inlet port (not shown) of the pump 110 is in fluid communication with the second inner cavity 206 via the channel 208. The second inner cavity 206 is further fluidly communicated with the wound via the bridge dressing 104 and the wound dressing 102. This would create a vacuum inside the casing 100 upon application of the negative pressure on the wound 52.

The casing 100 further includes a third portion 214 pivotally connected to the first portion 202 and pivotable relative to the first portion 202 independent of the second portion 210. The third portion 214 is configured to at least partially engage the first portion 202 in a closed position of the third portion 214. The third portion 214 covers the second inner cavity 206 in the closed position of the third portion 214, such that the second bridge end portion 108 of the bridge dressing 104 is received between the second inner cavity 206 and the third portion 214. The closed position of the third portion 214 is shown in FIG. 1. In FIGS. 2A to 2D, the closed position of the third portion 214 is not shown for illustrative purposes. Moreover, when the second bridge end portion 108 of the bridge dressing 104 is received between the second inner cavity 206 and the third portion 214, the material of the bridge dressing 104 prevents the collapse of the bridge dressing 104 in the closed position of the third portion 214.

In some embodiments, the system 50 further includes an electronics unit 112 (shown in FIG. 2A only for illustrative purposes) received within the first inner cavity 204 and the third inner cavity 212 in the closed position of the second portion 210. The electronics unit 112 may include a printed circuit board, wirings, and other electronic components to control operating parameters of the pump 110. In some embodiments, the casing 100 may include input buttons that can be accessed by a clinician to switch on and off the electronics unit 112. In some embodiments, the casing 100 may further include visual indicators to check various parameters associated with the electronics unit 112 (e.g., a status of the remaining battery power).

In some embodiments, the casing 100 further includes a first living hinge 216 (shown in FIG. 2B) pivotally connecting the first portion 202 to the second portion 210. The casing 100 further includes a second living hinge 218 (shown in FIG. 2B) separate from the first living hinge 216 and pivotally connecting the first portion 202 to the third portion 214. In some embodiments, the casing 100 further defines a gap G disposed between the second portion 210 and the third portion 214, such that the second portion 210 and the third portion 214 are pivotable independently of each other relative to the first portion 202. In other words, the third portion 214 is pivotally movable relative to the first portion 202 independent of the second portion 210. Similarly, the second portion 210 is pivotally movable relative to the first portion 202 independent of the third portion 214.

In some embodiments, an area of each of the first inner cavity 204 and the third inner cavity 212 is greater than an area of the second inner cavity 206. In some other embodiments, the area of each of the first inner cavity 204 and the third inner cavity 212 is less than or equal to the area of the second inner cavity 206. In some embodiments, the first inner cavity 204 and the third inner cavity 212 have similar shapes and dimensions. In some other embodiments, the first inner cavity 204 and the third inner cavity 212 may have different shapes and dimensions.

The first portion 202 further includes a first perimeter P1, a first inner surface 220, and a first outer surface 222 opposing the first inner surface 220. The first inner surface 224 is at least partially bounded by the first perimeter P1. In some embodiments, the first outer surface 222 can be interchangeably referred to herein as “an outer surface 222”. The first inner surface 220 of the first portion 202 at least partially engages with the second portion 210 in the closed position of the second portion 210 and the third portion 214 in the closed position of the third portion 214.

The second portion 210 further includes a second perimeter P2, a second inner surface 224, and a second outer surface 226 opposing the second inner surface 224. The second inner surface 224 is at least partially bounded by the second perimeter P2. The second inner surface 224 of the second portion 210 at least partially engages with the first portion 202 in the closed position of the second portion 210.

In some embodiments, the first portion 202 is adhesively bonded to the second portion 210 in the closed position of the second portion 210. In some cases, an adhesive may be applied to at least one of the first inner surface 220 of the first portion 202 and the second inner surface 224 of the second portion 210, such that the second inner surface 224 of the second portion 210 is kept engaged with the first inner surface 220 of the first portion 202 in the closed position of the second portion 210. The adhesive may be a pressure-sensitive adhesive.

In some embodiments, the first portion 202 is adhesively bonded to the third portion 214 in the closed position of the third portion 214. In some cases, an adhesive may be applied to at least one of the first inner surface 220 of the first portion 202 and the third portion 214, such that the third portion 214 is kept engaged with the first inner surface 220 of the first portion 202 in the closed position of the third portion 214. The adhesive may be a pressure sensitive-adhesive or a releasable adhesive.

As the second bridge end portion 108 of the bridge dressing 104 is received between the second inner cavity 206 and the third portion 214, the bridge dressing 104 may be cut to a desired length based on different application attributes and preferences of a patient. Moreover, the system 50 may be wearable with placement of the casing 100 near the wound 52.

In some embodiments, the casing 100 is a one-piece component made from a closed-cell foam. In an application, the casing 100 may be web processed from a soft closed cell foam material to reduce cost, thereby providing a soft housing for the pump 110. The soft material of the casing 100 may allow the casing 100 to conform around the second bridge end portion 108 of the bridge dressing 104.

In some embodiments, the first portion 202 further includes a sloped surface 228 sloping from the first inner surface 220 towards the first outer surface 222. The first portion 202 further includes a peripheral section 230 extending from the first inner surface 220 to the sloped surface 228. The peripheral section 230 at least partially surrounds the sloped surface 228. Specifically, the peripheral section 230 surrounds the sloped surface 228 from three sides of the sloped surface 228. In some embodiments, the first portion 202 further includes an intermediate section 232 disposed between the first inner cavity 204 and the second inner cavity 206. The intermediate section 232 at least partially defines the first inner surface 220.

In some embodiments, the first portion 202 further includes a plurality of first transverse recesses 234 disposed adjacent to the second inner cavity 206 opposite to the first inner cavity 204 and a plurality of first transverse ribs 236 alternating with the plurality of first transverse recesses 234. The second portion 210 further includes a plurality of second transverse recesses 238 spaced apart from the third inner cavity 212 and a plurality of second transverse ribs 240 alternating with the plurality of second transverse recesses 238. In the closed position of the third portion 214, the plurality of first transverse recesses 234 are aligned with the plurality of second transverse recesses 238 and at least partially receive the bridge dressing 104 therebetween. Specifically, in the closed position of the third portion 214, the plurality of first transverse recesses 234 and the plurality of second transverse recesses 238 receive the second bridge end portion 108 of the bridge dressing 104 therebetween. The presence of the plurality of first transverse recesses 234, the plurality of first transverse ribs 236, the plurality of second transverse recesses 238, and the plurality of second transverse ribs 240 may improve a conformability of the casing 100 around the bridge dressing 104.

In some embodiments, the channel 208 includes a first channel portion C1 disposed in fluid communication with the first inner cavity 204 and extending through the first portion 202 from the first inner cavity 204 to the first outer surface 222. The channel 208 further includes a second channel portion C2 (shown in FIG. 2B) disposed in fluid communication with the first channel portion C1 and extending towards the second inner cavity 206. The second channel portion C2 is disposed on the first outer surface 222 and inclined to the first channel portion C1. Thus, the casing 100 includes the channel 208 at least partially disposed on the outer surface 222 and fluidly communicating with the inner cavity 204. In some embodiments, the first channel portion C1 is perpendicular to the second channel portion C2.

The channel 208 further includes a third channel portion C3 disposed in fluid communication with the second channel portion C2 and extending through the first portion 202 towards the second inner cavity 206. The third channel portion C3 is inclined to the second channel portion C2. In some embodiments, the third channel portion C3 is perpendicular to the second channel portion C2. In some embodiments, the first channel portion C1 is parallel to the third channel portion C3. The channel 208 further includes a fourth channel portion C4 fluidly communicating the third channel portion C3 with the second inner cavity 206. The fourth channel portion C4 is inclined to the third channel portion C3. In some embodiments, the fourth channel portion C4 is perpendicular to the third channel portion C3. In some embodiments, the second channel portion C2 is parallel to the fourth channel portion C4. Further, the fourth channel portion C4 is a cut-out formed in the intermediate section 232 and disposed adjacent to the second inner cavity 206. Moreover, the fourth channel portion C4 is a cut-out formed in the peripheral section 230.

In some embodiments, the system 50 further includes a drape 242 (shown as transparent for illustrative purposes) bonded to the first outer surface 222 of the first portion 202 and configured to sealingly cover the second channel portion C2 of the channel 208. The drape 242 may be a flexible sheet or a sealing film to limit any fluid communication between ambient and the second channel portion C2 of the channel 208. The drape 242 may be adhesively bonded to the first outer surface 222 of the first portion 202.

In some embodiments, each of the first inner cavity 204 and the third inner cavity 212 includes a rectangular planar surface 244 and a rectangular peripheral section 246 extending from and surrounding the rectangular planar surface 244.

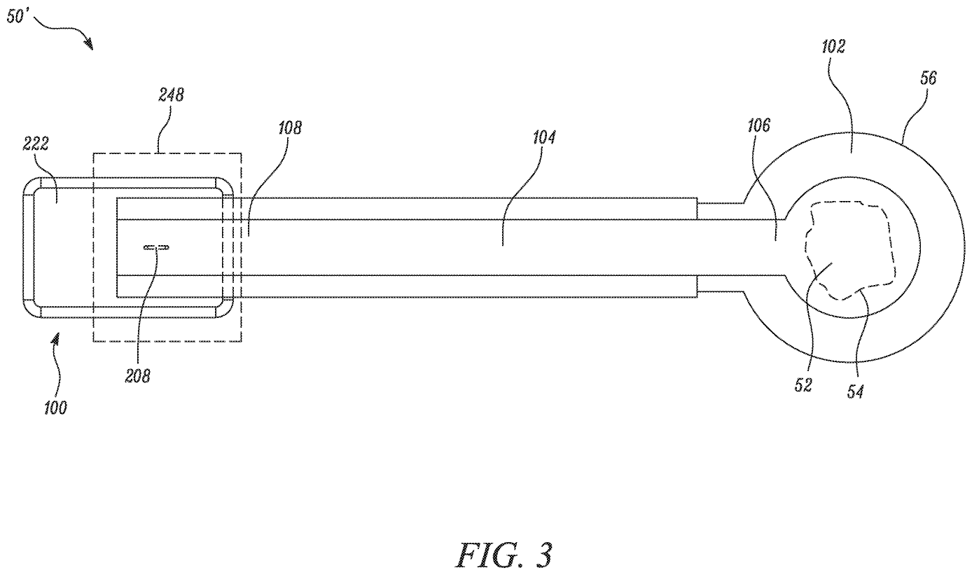

FIG. 3 is a schematic top view of a system 50′ for NPWT of the wound 52, according to another embodiment of the present disclosure. The system 50′ is substantially similar to the system 50 of FIG. 1, with common components being referred to by the same numerals. However, in the system 50′, the second bridge end portion 108 of the bridge dressing 104 is not received between the second inner cavity 206 and the third portion 214. In the system 50′, the second bridge end portion 108 of the bridge dressing 104 is connected to and disposed on the outer surface 222 of the casing 100, such that the second bridge end portion 108 fluidly communicates with and covers the channel 208 (also shown in FIG. 2B). Therefore, in the system 50′, the second bridge end portion 108 of the bridge dressing 104 is disposed on the outer surface 222 of the casing 100 and not received within the second inner cavity 206 of the first portion 202 of the casing 100.

In some embodiments, the system 50′ further includes a drape 248 adhesively bonding the second bridge end portion 108 to the outer surface 222 of the casing 100 and sealingly covering the second bridge end portion 108. The drape 248 may be an adhesive patch to adhesively bond the second bridge end portion 108 to the outer surface 222 of the casing 100 and also secure the casing 100 to body of a patient. Therefore, the system 50′ may be used in applications where the casing 100 is required to be placed on the body of the patient. The system 50′ may be comfortable for different patients as the casing 100 including the pump 110 could be adhered to the body of the patients.

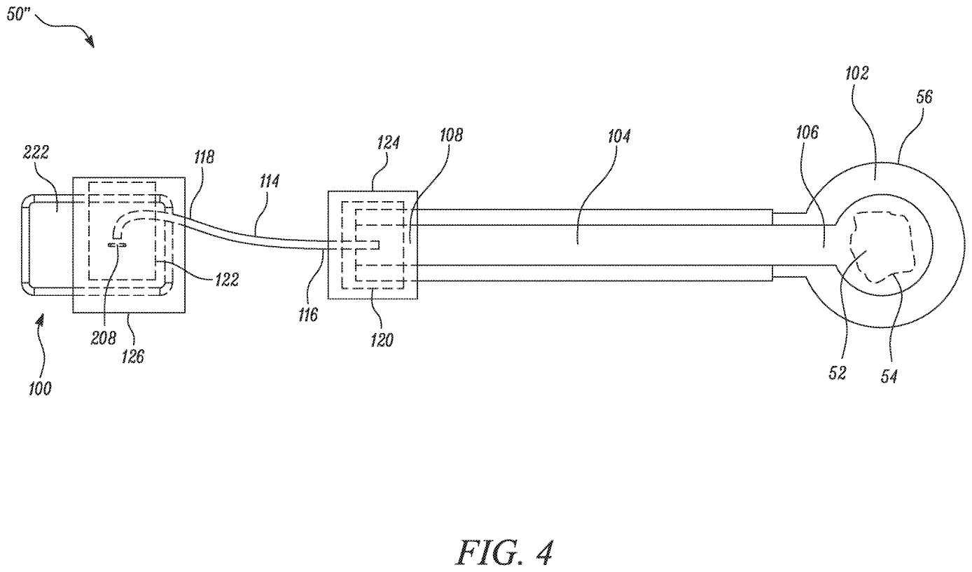

FIG. 4 is a schematic top view of a system 50″ for NPWT of the wound 52, according to another embodiment of the present disclosure. The system 50″ is substantially similar to the system 50′ of FIG. 3, with common components being referred to by the same numerals. However, in the system 50″, the second bridge end portion 108 of the bridge dressing 104 is not connected to and disposed on the outer surface 222 of the casing 100.

The system 50″ further includes a tube 114 including a first tube end portion 116 and a second tube end portion 118 opposite to the first tube end portion 116. The system 50″ further includes a first manifold 120 disposed around the first tube end portion 116 and fluidly coupled to the tube 114. The first manifold 120 is connected to the second bridge end portion 108 of the bridge dressing 104. The system 50″ further includes a second manifold 122 disposed around the second tube end portion 118 and fluidly coupled to the tube 114. The second manifold 122 is connected to and disposed on the outer surface 222 of the casing 100. The second manifold 122 fluidly communicates with and covers the channel 208. As each of the first manifold 120 and the second manifold 122 is fluidly coupled to the tube 114, the first manifold 120 is in an indirect fluid communication with the second manifold 122 via the tube 114. As the first manifold 120 is connected to the bridge dressing 104 and the second manifold 122 fluidly communicates with the channel 208, the bridge dressing 104 is in an indirect fluid communication with the channel 208 of the casing 100. Hence, the pump 110 can generate the negative pressure on the wound 52 through the channel 208, the first manifold 120, the second manifold, 122, the tube 114, the bridge dressing 104, and the wound dressing 102. Each of the first manifold 120 and the second manifold 122 may include a foam pad made from a soft injection molded material or web-processed soft closed cell foam.

In some embodiments, the system 50″ further includes a first adhesive film 124 adhesively bonding the first manifold 120 to the second bridge end portion 108 of the bridge dressing 104. The first adhesive film 124 sealingly covers the first manifold 120. The system 50″ further includes a second adhesive film 126 adhesively bonding the second manifold 122 to the outer surface 222 of the casing 100. The second adhesive film 126 sealingly covers the second manifold 122. The first manifold 120, the second manifold 122, the first adhesive film 124, and the second adhesive film 126 are shown as transparent in FIG. 4 for illustrative purposes.

The system 50″ may be beneficial for patients who desire remote placement of the casing 100 from their body, or for applications which require the remote placement of the casing 100 from the body of the patients. In some embodiments, the first manifold 120 and the second manifold 122 may be made from a soft injection molded material or web-processed soft closed cell foam. This may provide a positive patient experience.

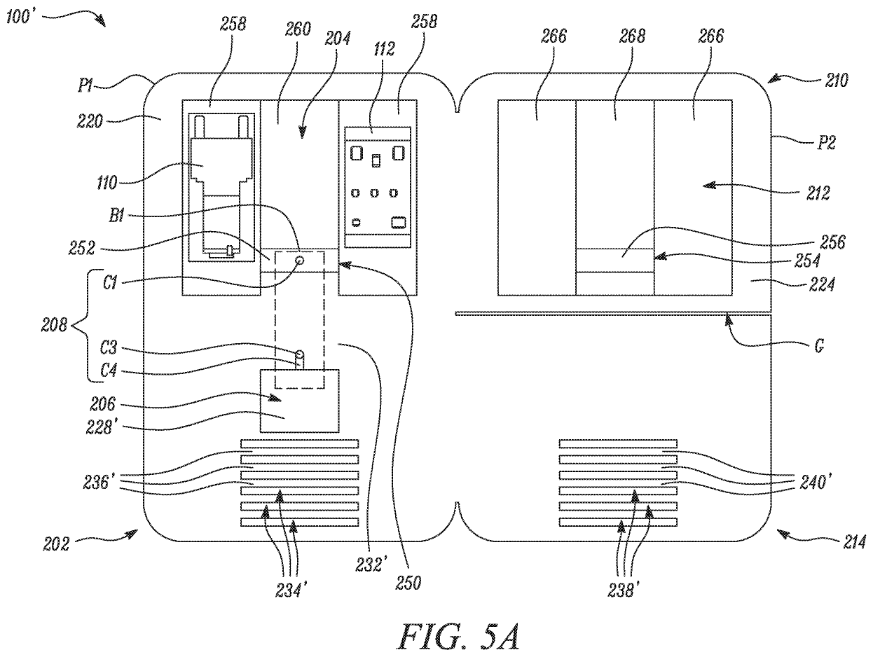

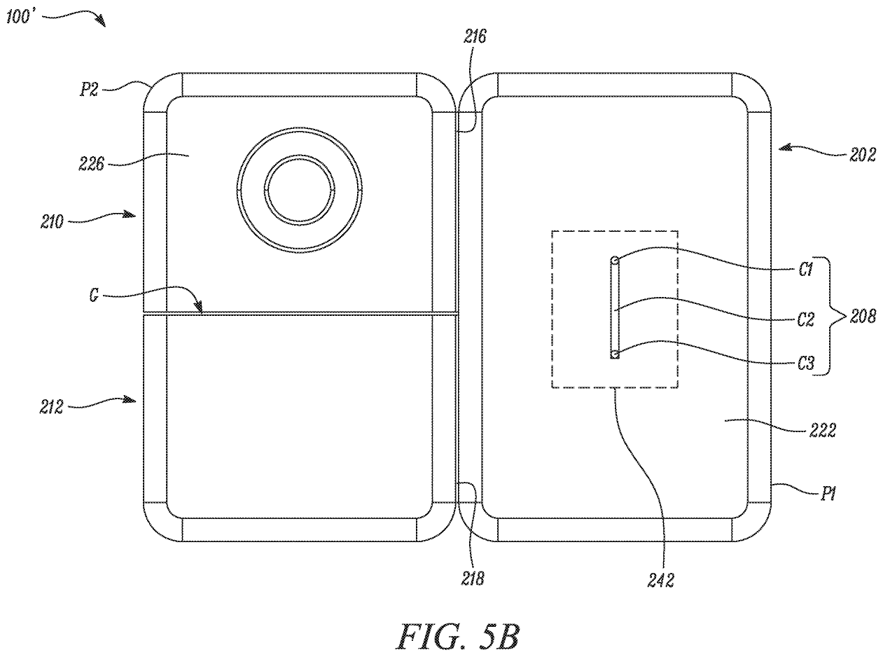

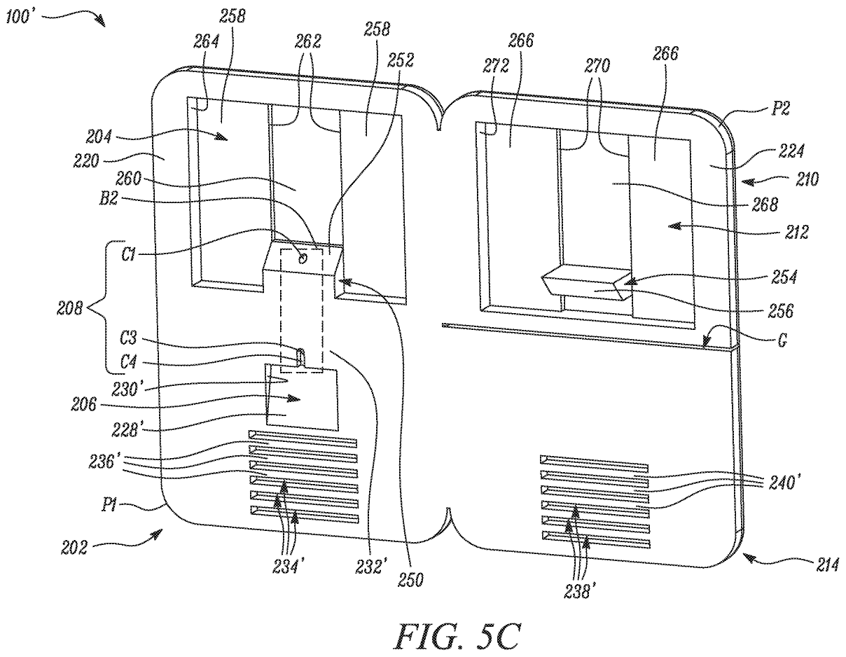

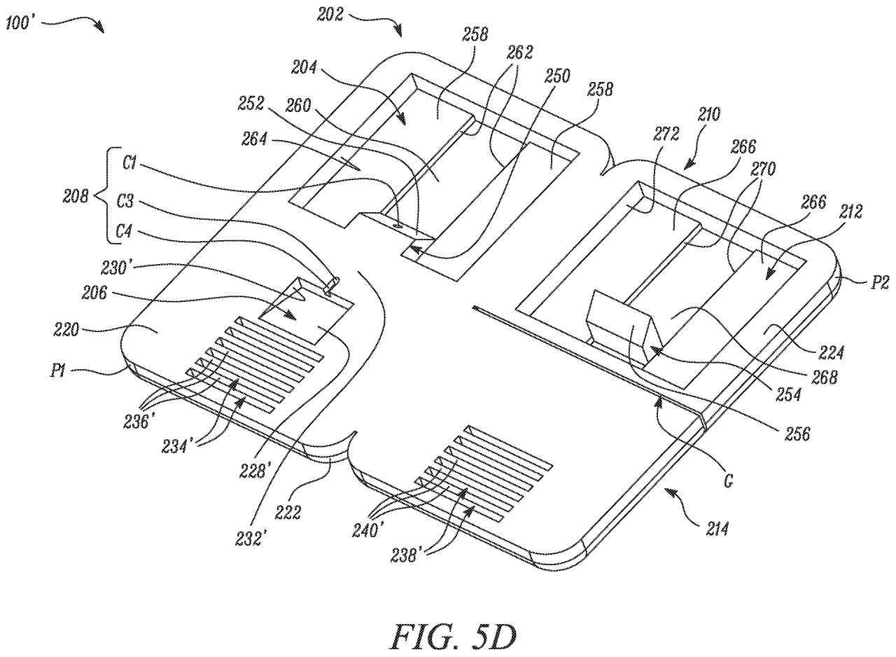

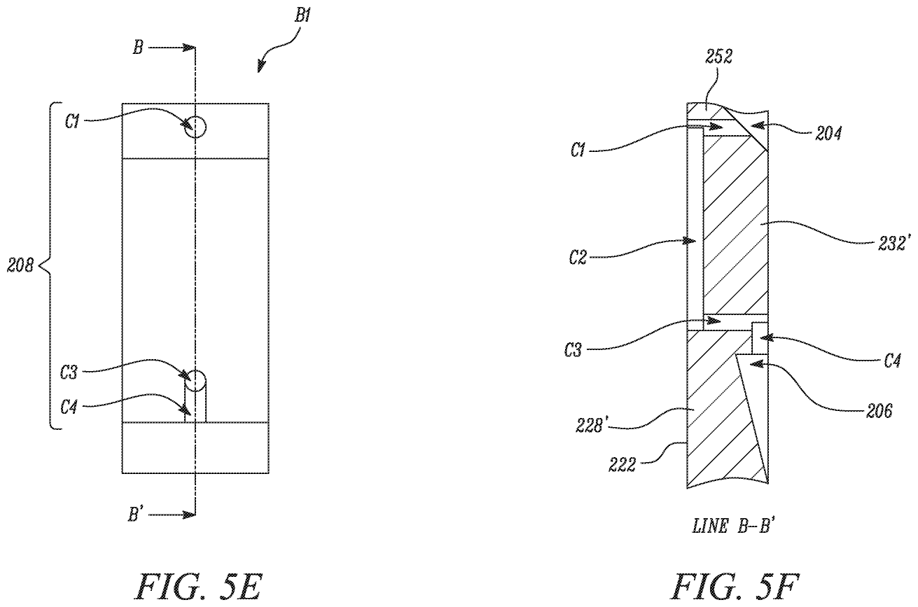

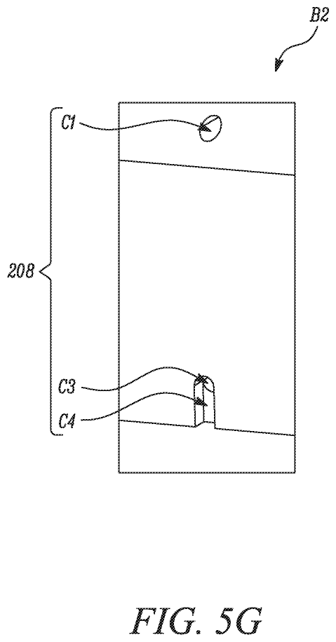

FIGS. 5A to 5D are different views of a casing 100′, according to another embodiment of the present disclosure. FIG. 5E is an enlarged view of a portion B1 (shown in FIG. 5A) of the casing 100′, according to an embodiment of the present disclosure. FIG. 5F is a side sectional view of the portion B1 along a line B-B′ shown in FIG. 5E, according to an embodiment of the present disclosure. FIG. 5G is an enlarged view of a portion B2 (shown in FIG. 5C) of the casing 100′, according to an embodiment of the present disclosure. The casing 100′ is substantially similar to the casing 100 shown in FIGS. 2A to 2D, with common components being referred to by the same numerals. A length of the second channel portion C2 (shown in FIG. 5B) of the casing 100′ is greater than a length of the second channel portion C2 of the casing 100 shown in FIG. 2B.

Referring to FIGS. 5A to 5G, in the casing 100′, the first portion 202 includes a sloped surface 228′ that is substantially similar to the sloped surface 228 (shown in FIG. 2C) of the casing 100. However, the sloped surface 228 of the casing 100 has different dimensions than the sloped surface 228′ of the casing 100′. Specifically, an area of the sloped surface 228′ is less than an area of the sloped surface 228. In the casing 100′, the first portion 202 further includes a peripheral section 230′ extending from the first inner surface 220 to the sloped surface 228′. The peripheral section 230′ at least partially surrounds the sloped surface 228′. In the casing 100′, the fourth channel portion C4 is a cut-out formed in the peripheral section 230′.

In the casing 100′, the first portion 202 further includes an intermediate section 232′ disposed between the first inner cavity 204 and the second inner cavity 206. The intermediate section 232′ at least partially defines the first inner surface 220. The intermediate section 232′ is substantially similar to the intermediate section 232 of the casing 100 shown in FIG. 2A. However, the intermediate section 232 of the casing 100 has different dimensions than the intermediate section 232′ of the casing 100′. Specifically, an area of the intermediate section 232 is less than an area of the intermediate section 232′.

In the casing 100′, the first portion 202 further includes a plurality of first transverse recesses 234′ disposed adjacent to the second inner cavity 206 opposite to the first inner cavity 204 and a plurality of first transverse ribs 236′ alternating with the plurality of first transverse recesses 234′. The plurality of first transverse recesses 234′ are substantially similar to the plurality of first transverse recesses 234 of the casing 100 shown in FIG. 2A. However, a length of the plurality of first transverse recesses 234′ is less than a length of the plurality of first transverse recesses 234 shown in FIG. 2A. Further, the plurality of first transverse ribs 236′ are substantially similar to the plurality of first transverse ribs 236 of the casing 100 shown in FIG. 2A. However, a length of the plurality of first transverse ribs 236′ is less than a length of the plurality of first transverse ribs 236 shown in FIG. 2A.

In the casing 100′, the second portion 210 further includes a plurality of second transverse recesses 238′ spaced apart from the third inner cavity 212 and a plurality of second transverse ribs 240′ alternating with the plurality of transverse recesses 238′. The plurality of second transverse recesses 238′ are substantially similar to the plurality of second transverse recesses 238 of the casing 100 shown in FIG. 2A. However, a length of the plurality of second transverse recesses 238′ is less than a length of the plurality of second transverse recesses 238 shown in FIG. 2A. Further, the plurality of second transverse ribs 240′ are substantially similar to the plurality of second transverse ribs 240 of the casing 100 shown in FIG. 2A. However, a length of the plurality of second transverse ribs 240′ is less than a length of the plurality of second transverse ribs 240 shown in FIG. 2A.

In some embodiments, the first portion 202 includes a first wedge 250 extending into the first inner cavity 204 and including a first wedge surface 252. The first channel portion C1 extends from the first wedge surface 252 at least partially through the first wedge 250. In other words, the first channel portion C1 extends from the first wedge surface 252 to the first outer surface 222.

In some embodiments, the second portion 210 further includes a second wedge 254 extending from the third inner cavity 212 and including a second wedge surface 256. The second wedge surface 256 is configured to lockingly engage with the first wedge surface 252 of the first wedge 250 in the closed position of the second portion 210. Engagement of the second wedge surface 256 with the first wedge surface 252 may isolate the second bridge end portion 108 of the bridge dressing 104 from the pump 110 to prevent any leakage through the pump 110 to the wound 52.

In the casing 100′, each of the first inner cavity 204 and the third inner cavity 212 does not include a rectangular planar surface (i.e., the rectangular planar surface 244 shown in FIG. 2A). In some embodiments, the first portion 202 further includes a pair of first raised surfaces 258 spaced apart from each other. The first portion 202 further includes a first lowered surface 260 extending between the pair of first raised surfaces 258. The first lowered surface 260 is disposed below the pair of first raised surfaces 258 with respect to the first inner surface 220. The first portion 202 further includes a pair of first connecting surfaces 262 (shown in FIG. 5C) corresponding to the pair of first raised surfaces 258. Each of the pair of first connecting surfaces 262 extends from the first lowered surface 260 to a corresponding first raised surface 258 from the pair of first raised surfaces 258. Therefore, the first lowered surface 260 and the pair of first connecting surfaces 262 may together form a U-shaped configuration. The first portion 202 further includes a first peripheral section 264 (shown in FIG. 5C) extending from and surrounding the pair of first raised surfaces 258. The first peripheral section 264 also surrounds the first lowered surface 260 and the pair of first connecting surfaces 262. The first peripheral section 264 further extends to the first inner surface 220. The first peripheral section 264 connects each of the pair of first raised surfaces 258 with the intermediate section 232′. The pair of first raised surfaces 258, the first lowered surface 360, the pair of first connecting surfaces 262, and the first peripheral section 264 together define the first inner cavity 204.

In some embodiments, the second portion 210 further includes a pair of second raised surfaces 266 spaced apart from each other. The second portion 210 further includes a second lowered surface 268 extending between the pair of second raised surfaces 266. The second lowered surface 268 is disposed below the pair of second raised surfaces 266 with respect to the second inner surface 224. The second portion 210 further includes a pair of second connecting surfaces 270 (shown in FIG. 5C) corresponding to the pair of second raised surfaces 266. Each of the pair of second connecting surfaces 270 extends from the second lowered surface 268 to a corresponding second raised surface 266 from the pair of second raised surfaces 266. Therefore, the second lowered surface 268 and the pair of second connecting surfaces 270 may together form a U-shaped configuration. The second portion 210 further includes a second peripheral section 272 (shown in FIG. 5C) extending from and surrounding the pair of second raised surfaces 266. The second peripheral section 272 also surrounds the second lowered surface 268 and the pair of second connecting surfaces 270. The second peripheral section 272 further extends to the second inner surface 224. The pair of second raised surfaces 266, the second lowered surface 268, the pair of second connecting surfaces 270, and the second peripheral section 272 together define the third inner cavity 212.

A functional advantage of the casing 100′ is the same as that of the casing 100 (shown in FIGS. 2A to 2D). In some embodiments, the system 50′ (shown in FIG. 3) may also include the casing 100′ (instead of the casing 100). In some embodiments, the system 50″ (shown in FIG. 4) may also include the casing 100′ (instead of the casing 100).

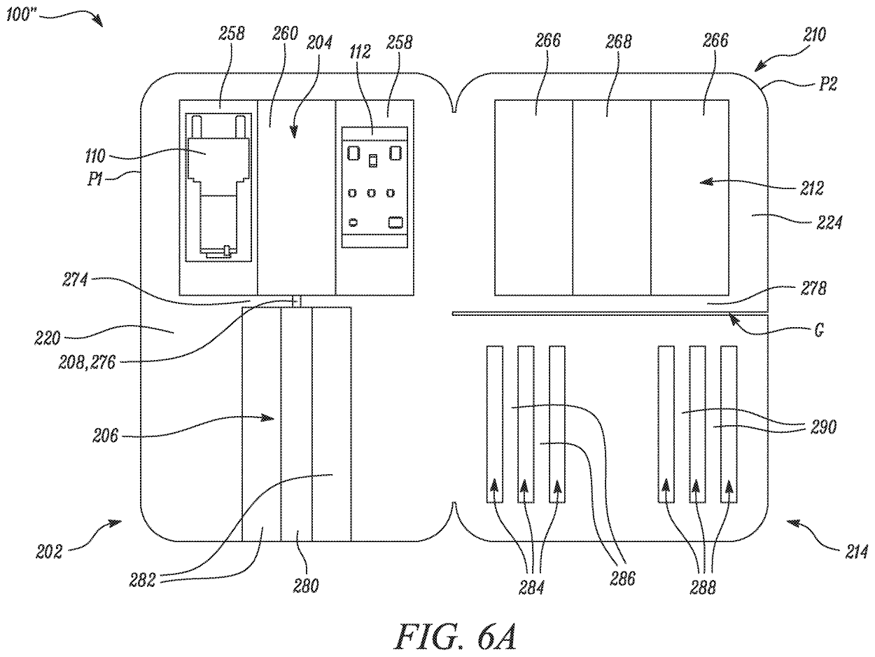

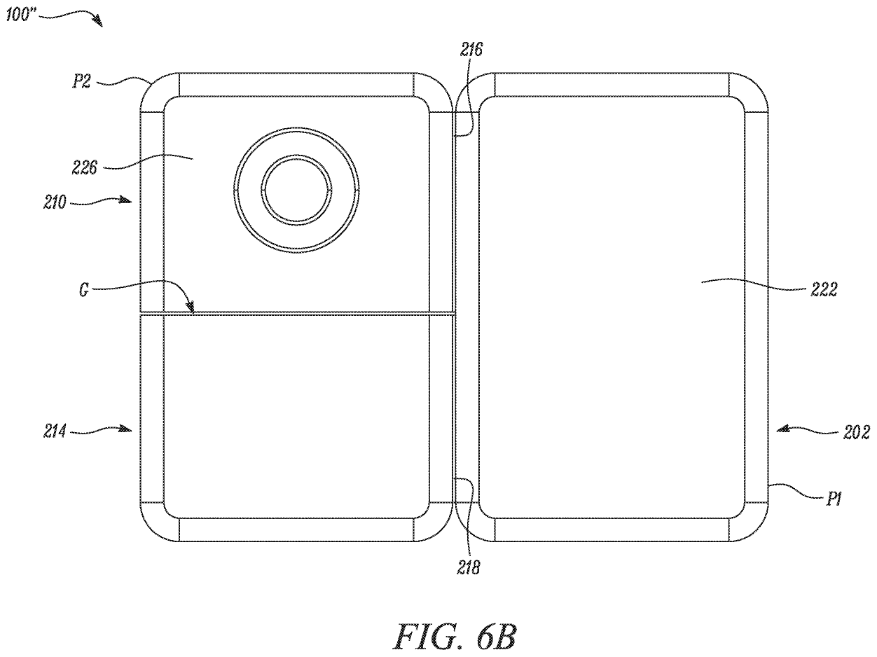

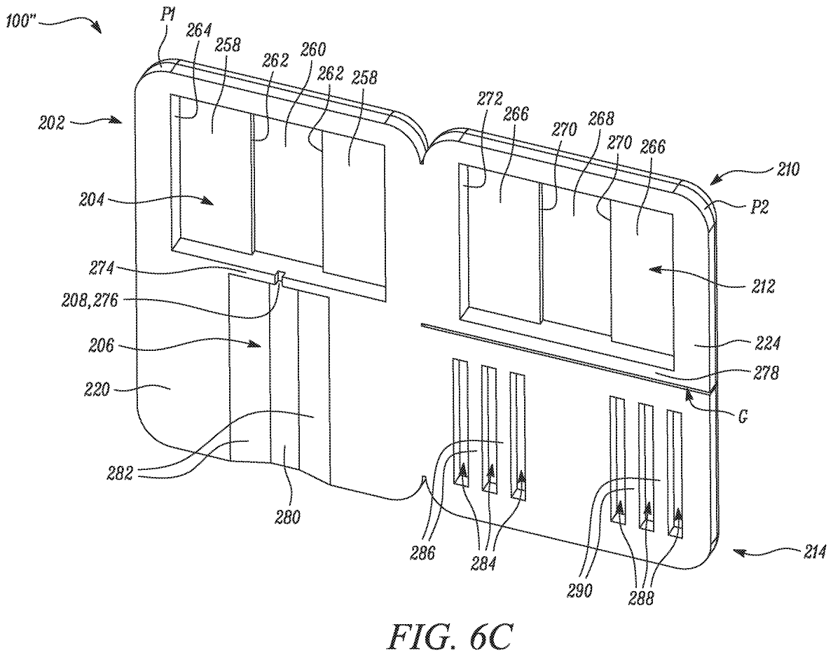

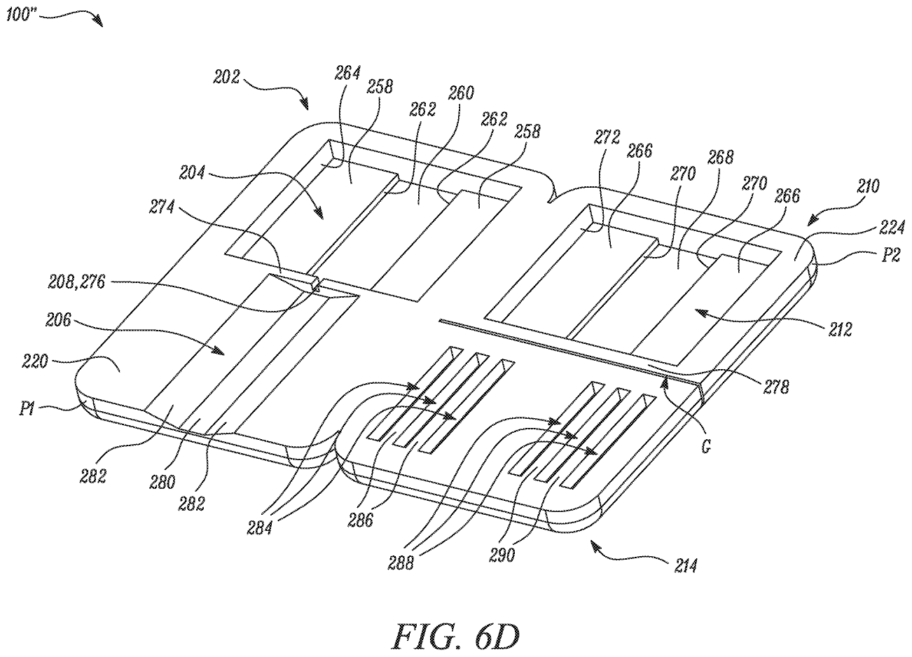

FIGS. 6A to 6D are different views of a casing 100″, according to another embodiment of the present disclosure. The casing 100″ is substantially similar to the casing 100′ shown in FIGS. 5A to 5D, with common components being referred to by the same numerals. However, in the casing 100″, the first portion 202 does not include a sloped surface (i.e., the sloped surface 228′ shown in FIG. 5C), an intermediate section (i.e., the intermediate section 232′ shown in FIG. 5C), different channel portions (shown in FIGS. 5A to 5G) of the channel 208, any wedge (i.e., the first wedge 250 and the second wedge 254), a drape (i.e., the drape 242 shown in FIG. 5B), a plurality of first transverse recesses (i.e., the plurality of first transverse recesses 234′ shown in FIG. 5C), a plurality of first transverse ribs (i.e., the plurality of first transverse ribs 236′ shown in FIG. 5C), a plurality of second transverse recesses (i.e., the plurality of second transverse recesses 238′ shown in FIG. 5C), and a plurality of second transverse ribs (i.e., the plurality of second transverse ribs 240′ shown in FIG. 5C).

In the casing 100″, the first portion 202 further includes a first wall 274 separating the first inner cavity 204 from the second inner cavity 206. The pair of first raised surfaces 258 extend from the first wall 274 towards the first perimeter P1 of the first portion 202 opposite to the second inner cavity 206. Further, the first lowered surface 260 extends from the first wall 274 towards the first perimeter P1 of the first portion 202 opposite to the second inner cavity 206. Further, in the casing 100″, the channel 208 includes an open slot 276 extending through the first wall 274 from the first inner cavity 204 to the second inner cavity 206. The channel 208 is internal as the open slot 276 extends through the first wall 274. As there is no channel portion disposed on the outer surface 222 (i.e., the channel 208 is internal), the casing 100″ may not be incorporated in the system 50′ (shown in FIG. 3).

In some embodiments, the second portion 210 further includes a second wall 278 disposed adjacent to the third inner cavity 212 and configured to engage with the first wall 274 in the closed position of the second portion 210. The open slot 276 is covered by the second wall 278 in the closed position of the second portion 210.

In some embodiments, the first portion 202 further includes a planar surface 280 extending from the first wall 274 to the first perimeter P1 of the first portion 202 opposite to the first inner cavity 204. In some embodiments, the planar surface 280 is a rectangular planar surface. The first portion 202 further includes a pair of inclined surfaces 282 extending from opposing sides of the planar surface 280 and inclined to the planar surface 280. Each of the pair of inclined surfaces 282 extends from the first wall 274 to the first perimeter P1 of the first portion 202 opposite to the first inner cavity 204. The planar surface 280, the pair of inclined surfaces 282, and the first wall 274 of the first portion 202 together define the second inner cavity 206. The second inner cavity 206 is open at the first perimeter P1 opposite to the first wall 274. As the second inner cavity 206 is open at the first perimeter P1, the second bridge end portion 108 of the bridge dressing 104 may be easily inserted and received between the second inner cavity 206 and the third portion 214.

In some embodiments, the third portion 214 includes a plurality of first longitudinal recesses 284 spaced apart from the third inner cavity 212 and a plurality of first longitudinal ribs 286 alternating with the plurality of first longitudinal recesses 284. The plurality of first longitudinal recesses 284 are disposed proximal to the second living hinge 218 (shown in FIG. 6B). The third portion 214 further includes a plurality of second longitudinal recesses 288 spaced apart from the third inner cavity 212 and a plurality of second longitudinal ribs 290 alternating with the plurality of second longitudinal recesses 288. The plurality of second longitudinal recesses 288 are disposed distal to the second living hinge 218. Further, at least one of the plurality of first longitudinal recesses 284 and at least one of the plurality of second longitudinal recesses 288 at least partially engage the bridge dressing 104 in the closed position of the third portion 214.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A system for negative pressure wound therapy (NPWT) of a wound, the system comprising:
a bridge dressing comprising a first bridge end portion proximal to the wound and a second bridge end portion opposite to the first bridge end portion;
a pump; and
a casing spaced apart from the wound, the casing comprising:
a first portion comprising a first inner cavity configured to at least partially receive the pump therein, a second inner cavity spaced apart from the first inner cavity and configured to receive the second bridge end portion of the bridge dressing therein, and a channel fluidly communicating the first inner cavity with the second inner cavity;
a second portion pivotally connected to the first portion and configured to at least partially engage the first portion in a closed position of the second portion, the second portion comprising a third inner cavity that is aligned with the first inner cavity in the closed position of the second portion, such that the pump is received within the first inner cavity and the third inner cavity; and
a third portion pivotally connected to the first portion and pivotable relative to the first portion independent of the second portion, wherein the third portion is configured to at least partially engage the first portion in a closed position of the third portion, wherein the third portion covers the second inner cavity in the closed position of the third portion, such that the second bridge end portion of the bridge dressing is received between the second inner cavity and the third portion.

2. The system of claim 1, wherein the first portion further comprises:
a first perimeter;
a first inner surface and a first outer surface opposing the first inner surface, wherein the first inner surface of the first portion at least partially engages with the second portion in the closed position of the second portion and the third portion in the closed position of the third portion;
a first wall separating the first inner cavity from the second inner cavity;
a pair of first raised surfaces spaced apart from each other and extending from the first wall towards the first perimeter of the first portion opposite to the second inner cavity;
a first lowered surface extending from the first wall towards the first perimeter of the first portion opposite to the second inner cavity, wherein the first lowered surface further extends between the pair of first raised surfaces and is disposed below the pair of first raised surfaces with respect to the first inner surface;
a pair of first connecting surfaces corresponding to the pair of first raised surfaces, wherein each of the pair of first connecting surfaces extends from the first lowered surface to a corresponding first raised surface from the pair of first raised surfaces; and
a first peripheral section extending from and surrounding the pair of first raised surfaces, the first lowered surface, and the pair of first connecting surfaces, wherein the first peripheral section further extends to the first inner surface;
wherein the pair of first raised surfaces, the first lowered surface, the pair of first connecting surfaces, and the first peripheral section together define the first inner cavity.

3. The system of claim 2, wherein the channel comprises an open slot extending through the first wall from the first inner cavity to the second inner cavity.

4. The system of claim 3, wherein the second portion further comprises a second wall disposed adjacent to the third inner cavity and configured to engage with the first wall in the closed position of the second portion, and wherein the open slot is covered by the second wall in the closed position of the second portion.

5. The system of claim 1, wherein the first portion further comprises:
a first perimeter;
a first wall separating the first inner cavity from the second inner cavity;
a planar surface extending from the first wall to the first perimeter of the first portion opposite to the first inner cavity; and
a pair of inclined surfaces extending from opposing sides of the planar surface and inclined to the planar surface, wherein each of the pair of inclined surfaces extends from the first wall to the first perimeter of the first portion opposite to the first inner cavity;
wherein the planar surface, the pair of inclined surfaces, and the first wall of the first portion together define the second inner cavity, and wherein the second inner cavity is open at the first perimeter opposite to the first wall.

6. The system of claim 1, wherein the second portion further comprises:
a second perimeter;
a second inner surface and a second outer surface opposing the second inner surface, wherein the second inner surface of the second portion at least partially engages with the first portion in the closed position of the second portion;
a pair of second raised surfaces spaced apart from each other;
a second lowered surface extending between the pair of second raised surfaces, wherein the second lowered surface is disposed below the pair of second raised surfaces with respect to the second inner surface;
a pair of second connecting surfaces corresponding to the pair of second raised surfaces, wherein each of the pair of second connecting surfaces extends from the second lowered surface to a corresponding second raised surface from the pair of second raised surfaces; and
a second peripheral section extending from and surrounding the pair of second raised surfaces, the second lowered surface, and the pair of second connecting surfaces, wherein the second peripheral section further extends to the second inner surface;
wherein the pair of second raised surfaces, the second lowered surface, the pair of second connecting surfaces, and the second peripheral section together define the third inner cavity.

7. The system of claim 1, wherein the casing further comprises:
a first living hinge pivotally connecting the first portion to the second portion; and
a second living hinge separate from the first living hinge and pivotally connecting the first portion to the third portion.

8. The system of claim 7, wherein the third portion comprises:
a plurality of first longitudinal recesses spaced apart from the third inner cavity and a plurality of first longitudinal ribs alternating with the plurality of first longitudinal recesses, wherein the plurality of first longitudinal recesses are disposed proximal to the second living hinge; and
a plurality of second longitudinal recesses spaced apart from the third inner cavity and a plurality of second longitudinal ribs alternating with the plurality of second longitudinal recesses, wherein the plurality of second longitudinal recesses are disposed distal to the second living hinge;
wherein at least one of the plurality of first longitudinal recesses and at least one of the plurality of second longitudinal recesses at least partially engage the bridge dressing in the closed position of the third portion.

9. The system of claim 1, further comprising an electronics unit received within the first inner cavity and the third inner cavity in the closed position of the second portion.

10. The system of claim 1, wherein the casing further defines a gap disposed between the second portion and the third portion, such that the second portion and the third portion are pivotable independently of each other relative to the first portion.

11.-13. (canceled)

14. The system of claim 1, wherein the first portion further comprises:
a first inner surface and a first outer surface opposing the first inner surface, wherein the first inner surface of the first portion at least partially engages with the second portion in the closed position of the second portion and the third portion in the closed position of the third portion; and
a sloped surface sloping from the first inner surface towards the first outer surface and a peripheral section extending from the first inner surface to the sloped surface, wherein the peripheral section at least partially surrounds the sloped surface.

15. The system of claim 14, wherein the channel comprises:
a first channel portion disposed in fluid communication with the first inner cavity and extending through the first portion from the first inner cavity to the first outer surface;
a second channel portion disposed in fluid communication with the first channel portion and extending towards the second inner cavity, wherein the second channel portion is disposed on the first outer surface and inclined to the first channel portion;
a third channel portion disposed in fluid communication with the second channel portion and extending through the first portion towards the second inner cavity, wherein the third channel portion is inclined to the second channel portion; and
a fourth channel portion fluidly communicating the third channel portion with the second inner cavity, wherein the fourth channel portion is inclined to the third channel portion.

16. The system of claim 15, wherein the first channel portion is parallel to the third channel portion, wherein the first channel portion is perpendicular to the second channel portion, and wherein the second channel portion is parallel to the fourth channel portion.

17. The system of claim 15, wherein the first portion further comprises an intermediate section disposed between the first inner cavity and the second inner cavity, wherein the intermediate section at least partially defines the first inner surface, and wherein the fourth channel portion is a cut-out formed in the intermediate section and disposed adjacent to the second inner cavity.

18. The system of claim 15, wherein the first portion further comprises a first wedge extending into the first inner cavity and comprising a first wedge surface, and wherein the first channel portion extends from the first wedge surface at least partially through the first wedge.

19. The system of claim 18, wherein the second portion further comprises a second wedge extending from the third inner cavity and comprising a second wedge surface, and wherein the second wedge surface is configured to lockingly engage with the first wedge surface of the first wedge in the closed position of the second portion.

20. The system of claim 15, further comprising a drape bonded to the first outer surface of the first portion and configured to sealingly cover the second channel portion of the channel.

21. The system of claim 1, wherein the first portion further comprises a plurality of first transverse recesses disposed adjacent to the second inner cavity opposite to the first inner cavity and a plurality of first transverse ribs alternating with the plurality of first transverse recesses, wherein the second portion further comprises a plurality of second transverse recesses spaced apart from the third inner cavity and a plurality of second transverse ribs alternating with the plurality of transverse recesses, wherein, in the closed position of the third portion, the plurality of first transverse recesses are aligned with the plurality of second transverse recesses and at least partially receive the bridge dressing therebetween.

22.-26. (canceled)

27. A system for negative pressure wound therapy (NPWT) of a wound, the system comprising:
a bridge dressing comprising a first bridge end portion proximal to the wound and a second bridge end portion opposite to the first bridge end portion;
a pump; and
a casing spaced apart from the wound, the casing comprising an inner cavity receiving the pump therein, an outer surface, and a channel at least partially disposed on the outer surface and fluidly communicating with the inner cavity;
wherein the second bridge end portion of the bridge dressing is connected to and disposed on the outer surface of the casing, and wherein the second bridge end portion fluidly communicates with and covers the channel.

28. The system of claim 27, further comprising a drape adhesively bonding the second bridge end portion to the outer surface of the casing and sealingly covering the second bridge end portion.

29.-31. (canceled)

32. A system for negative pressure wound therapy (NPWT) of a wound, the system comprising:
a bridge dressing comprising a first bridge end portion proximal to the wound and a second bridge end portion opposite to the first bridge end portion;
a tube comprising a first tube end portion and a second tube end portion opposite to the first tube end portion;
a first manifold disposed around the first tube end portion and fluidly coupled to the tube, wherein the first manifold is connected to the second bridge end portion of the bridge dressing;
a second manifold disposed around the second tube end portion and fluidly coupled to the tube;
a pump; and
a casing spaced apart from the wound, the casing comprising an inner cavity receiving the pump therein, an outer surface, and a channel at least partially disposed on the outer surface and fluidly communicating with the inner cavity;
wherein the second manifold is connected to and disposed on the outer surface of the casing, and wherein the second manifold fluidly communicates with and covers the channel.

33.-36. (canceled)
